(19) United States
(12) Patent Application Publication
(10) Pub. No.: US 2010/0317078 A1
(43) Pub. Date: Dec. 16, 2010

(54) METHODS TO IMPROVE ALCOHOL TOLERANCE OF MICROORGANISMS

(75) Inventors: Manuel J. Villa-Garcia, Ithaca, NY (US); Erin J. Krause, Baldwinsville, NY (US); Larry P. Walker, Ithaca, NY (US); Susan A. Henry, Ithaca, NY (US)

Correspondence Address: NIXON PEABODY LLP - PATENT GROUP, 1100 CLINTON SQUARE, ROCHESTER, NY, 14604, US

(73) Assignee: Cornell Research Foundation Inc

(21) Appl. No.: 12/675,881

(22) PCT Filed: Aug. 27, 2008
(86) PCT No.: PCT/US08/74419

§ 371 Date: Aug. 30, 2010

Related U.S. Application Data
(60) Provisional application No. 60/968,247, filed on Aug. 27, 2007.

(51) Int. Cl. C12P 7/06; C12N 1/16; C12N 15/81; C12P 7/02

(52) U.S. Cl. 435/161; 435/255.1; 435/255.4; 435/255.5; 435/255.2; 435/471; 435/155

(57) ABSTRACT

The present invention is directed to a method of producing organisms tolerant to alcohol, that includes selecting a microorganism needing tolerance to alcohol and modifying the selected microorganism under conditions effective to overproduce inositol by the microorganism compared to when the microorganism is not modified, with the modified microorganism being tolerant to alcohol. The present invention is also directed to a method of producing alcohol that includes providing a microorganism tolerant to alcohol which is modified to overproduce inositol by the microorganism compared to when the microorganism is not modified. A fermentable feedstock is treated with the modified microorganism under conditions effective to produce the alcohol. The modified microorganism is also able to produce and tolerate alcohol in high osmolarity feedstocks.

This application claims the benefit of U.S. Provisional Patent Application Ser. No. 60/968,247, filed Aug. 27, 2007, which is hereby incorporated by reference in its entirety.

The subject matter of this application was made with support from the United States Government under National Institutes of Health (NIH) Grant No. RO1-GM019629-35 and under the United States Department of Agriculture (USDA) Grant No. 2001-52104-11484. The U.S. government has certain rights.

### FIELD OF THE INVENTION

The present invention is directed to methods to improve alcohol tolerance of microorganisms.

### BACKGROUND OF THE INVENTION

The continued evolution of a domestic bio fuels industry hinges on expanding our understanding of microbial fermentation of sugars derived from starchy or lignocellulosic biomass. Countless microorganisms are capable of this task and a handful of yeast excel at the conversion of sugars into ethanol. The yeast Saccharomyces cerevisiae is the most widely used of all yeast strains for the ethanol fuel industry. As with many microorganisms, the production and accumulation of certain metabolites, such as ethanol, can have a detrimental effect on cell growth and productivity.

Of chief concern to the ethanol fuel industry is the maximum concentration of ethanol that S. cerevisiae and other microorganisms can tolerate and remain productive. Increasing this threshold concentration is critical for the economic feasibility of large-scale bio fuel production facilities. Success in achieving higher ethanol concentrations during fermentation lessens the energy required to separate the ethanol and water fractions of the fermentation broth, a process that has an exponentially increasing energy requirement with decreasing ethanol concentration. Increasing the ethanol concentration in the fermentation product from 5% to 14% reduces the energy requirement for distillation by approximately 50% (Jacques et al., The Alcohol Textbook, 3^{rd} ed. Nottingham Press, Nottingham, United Kingdom, (1999)). Thus, expanding the understanding of the molecular mechanisms that confer ethanol tolerance for yeast and other microorganisms is an important goal of the industrial biotechnology community.

The plasma membrane—the active barrier between the cytoplasm and the extracellular environment—is thought to be the primary target of ethanol damage for yeast and other microorganisms (Beaven et al., “Production and Tolerance of Ethanol in Relation to Phospholipid Fatty Acyl Composition in Saccharomyces cerevisiae NCYC 431,” J Gen Microbiology 128:1447-1455 (1982); Casey et al., “Ethanol Tolerance in Yeasts,” CRC Crit Rev Microbiology 13(3):219-280 (1986); D'Amore et al., “A Study of Ethanol Tolerance in Yeast,” Crit Rev Biotechnology 9(4):287-304 (1990); Ingram et al., “Effects of Alcohols on Microorganisms,” Adv Microbial Phys 25:253-300 (1984), Mansure et al., “Trehalose Inhibits Ethanol Effects on Intact Yeast Cells and Liposomes,” Biochimica Biophysica Acta 1191:309-316 (1994), and Thomas et al., “Inhibitory Effect of Ethanol on Growth and Solute Accumulation by Saccharomyces cerevisiae as Affected by Plasma-membrane Lipid Composition,” Archives Microbiology 122:49-55 (1979)). Upon intercalation into the hydrophobic region of the cell membrane, ethanol opens the membrane structure, creating a more fluid environment and making the membrane more permeable to polar molecules. This process weakens hydrophobic interactions and affects the position and functionality of integral membrane proteins (Ingram et al., “Effects of Alcohols on Microorganisms,” Adv Microbial Phys 25:253-300 (1984)). Intracellular membranes, most notably the mitochondrial membrane, are thought to be similarly affected by ethanol (Chi et al., “Saccharomyces cerevisiae Strains with Different Degrees of Ethanol Tolerance Exhibit Different Adaptive Responses to Produced Ethanol,” J Industrial Microbiology Biotechnology 24:75-78 (2000)). Through the increased permeability of these barriers, ethanol has the ability to directly or indirectly disrupt critical cellular processes such as nutrient transport, proton flux, and virtually every process that takes places within or across the membrane walls.

Changes in phospholipid composition clearly play a role in increasing the ability of yeast to tolerate ethanol (Chi et al., “Role of Phosphatidylinositol (PI) in Ethanol Production and Ethanol Tolerance By a High Ethanol Producing Yeast,” J Industrial Microbiology and Biotechnology 22:58-63 (1999); Chi et al., Saccharomyces cerevisiae Strains with Different Degrees of Ethanol Tolerance Exhibit Different Adaptive Responses to Produced Ethanol,” J Industrial Microbiology Biotechnology 24:75-78 (2000); Furukawa et al., “Effect of Cellular Inositol Content on Ethanol Tolerance of Saccharomyces cerevisiae in Sake Brewing,” J Bioscience Bioengineering 98(2):107-113 (2004), and Jones et al., “Ethanol and the Fluidity of the Yeast Plasma Membrane,” Yeast 3:223-232 (1987)). The composition of the major phospholipids in the plasma membrane of the yeast cell is, in general, well defined. Major phospholipids of yeast cells, depending on the growth regime, are phosphatidylcholine (PC), phosphatidylethanolamine (PE), phosphatidylinositol (PI), and lesser quantities of phosphatidylserine (PS), phosphatidylglycerol and cardiolipin. The ionically charged hydrophilic head groups of phospholipids impart unique packing geometries in the plasma membrane. Yeast cells are capable of altering the relative proportion of each phospholipid in response to environmental stressors (Greenberg et al., “Genetic Regulation of Phospholipid Biosynthesis in Saccharomyces cerevisiae,” Microbiological Reviews 60(1):1-20 (1996)). It has been suggested that altering the ratio of charged head groups can affect ethanol tolerance. Clark and Beard (Clark et al., “Altered Phospholipids Composition in Mutants of Escherichia coli Sensitive or Resistant to Organic Solvents,” J Gen Microbiology 113:267-274 (1979)) found that decreasing the ratio of anionic:zwitterionic phospholipids in E. coli, by increasing PE content rendered yeasts cells less tolerant to organic solvents. Additional reports find reduced ATPase inhibition by ethanol in cells containing higher proportions of PS (Thomas et al., “Inhibitory Effect of Ethanol on Growth and Solute Accumulation by Saccharomyces cerevisiae as Affected by Plasma-membrane Lipid Composition,” Archives Microbiology 122:49-55 (1979)) or PI (Furukawa et al., “Effect of Cellular Inositol Content on Ethanol Tolerance of Saccharomyces cerevisiae in Sake Brewing,” J Bioscience Bioengineering 98(2):107-113 (2004)).

Yeast cells containing a higher PI concentration in the cellular membrane, due to inositol supplementation in the growth media, have been shown to tolerate and produce higher ethanol concentrations (Chi et al., “Role of Phosphatidylinositol (PI) in Ethanol Production and Ethanol Tolerance By a High Ethanol Producing Yeast,” J Industrial Microbiology and Biotechnology 22:58-63 (1999); and Furukawa et al., “Effect of Cellular Inositol Content on Ethanol Tolerance of Saccharomyces cerevisiae in Sake Brewing,” J Bioscience Bioengineering 98(2):107-113 (2004)). One hypothesis that can be drawn from these studies is that increasing the proportion of PI in the cellular membrane of S. cerevisiae will enhance its ethanol tolerance. Inositol supplementation in growth media has reportedly caused an increase in the rate of PI synthesis as well as the cellular PI content in wild type cells (Gaspar et al., “Inositol Induces a profound Alteration in the Pattern and Rate of Synthesis and Turnover of Membrane Lipids in Saccharomyces cerevisiae,” J Biol Chem 281(32):22773-22785 (2006), Greenberg et al., “Genetic Regulation of Phospholipid Biosynthesis in Saccharomyces cerevisiae,” Microbiological Reviews 60(1):1-20 (1996); Jiranek et al., “Pleiotropic Effects of the opi1 Regulatory Mutation of Yeast: its Effects on Growth and on Phospholipid and Inisitol Metabolism,” Microbiology 144:2739-2748 (1998); and White et al., “Inositol Metabolism in Yeasts,” Adv Microbial Phys 32:1-51 (1991)).

The present invention is directed to overcoming the deficiencies in the art.

### SUMMARY OF THE INVENTION

A first aspect of the present invention relates to a method of producing organisms tolerant to alcohol that includes selecting a microorganism needing tolerance to alcohol and modifying the selected microorganism under conditions effective to overproduce inositol by the microorganism compared to when the microorganism is not modified, with the modified microorganism being tolerant to alcohol.

A second aspect of the present invention relates to a method of producing alcohol that includes providing a microorganism that is tolerant to alcohol and modified to overproduce inositol compared to that when the microorganism is not modified. The method comprises the use of a fermentable feedstock, which is treated with the modified microorganism under conditions effective to produce the alcohol.

The present invention assesses the effects of inositol supplementation in normal and high osmolarity growth media as well as to compare the ethanol tolerance of the wild type S. cerevisiae to an opi1 strain (the opi strain presents an Opi− phenotype or overproduction of inositol). The OPI1 gene product is a negative regulatory factor that controls the transcription of the structural gene INO1, which encodes the enzyme catalyzing the limiting step in the biosynthesis of inositol, the conversion of glucose-6-phosphate to inositol-3-phosphate. A dephosphorylation of inositol-3-phosphate by another enzyme completes the inositol biosynthesis. Upon the deletion of the OPI1 gene, the cell will constitutively produce inositol, regardless of the extracellular inositol concentration. The opi1 strain has been shown to accumulate higher levels of PI in the cellular membrane without media supplementation (Jiranek et al., “Pleiotropic Effects of the opi1 Regulatory Mutation of Yeast: its Effects on Growth and on Phospholipid and Inisitol Metabolism,” Microbiology 144:2739-2748 (1998), which is hereby incorporated by reference in its entirety). Experimental studies designed to measure and compare the effects of inositol supplementation and the effects of the opi1 mutation on ethanol tolerance of S. cerevisiae grown in the presence of exogenous ethanol are demonstrated here.

However, supplementation of inositol to the fermentation media to increase PI in the cell membranes and effectively increasing tolerance to high alcohol concentrations is not cost effective at an industrial scale. Therefore, genetic modification of microorganisms to increase de novo biosynthesis of inositol and inositol containing molecules such as PI is the key to practically increase tolerance to high alcohol concentrations and increase the microorganism fermentation capacity. Furthermore, given the industrial use of high osmolarity feedstocks (or fermentation media) to produce alcohol by fermentation, the microorganism of choice for this process must have in addition to an increased alcohol tolerance, an increase tolerance to osmotic shock. A high osmolarity feedstock may contain high sugar, high salts, high solids concentrations, or a combination of the three. Microorganisms grown in such high osmolarity media undergo an osmotic shock that may lyse the cell or reduces their normal growth rate and fermentation capacity. The microorganism, if it is able to survive the osmotic shock, mounts a high osmolarity stress response by producing metabolites such as glycerol and trehalose which may reduce the final alcohol yield of the fermentation

### BRIEF DESCRIPTION OF THE DRAWINGS

FIG. 1 shows cell viability of wild type and opi1 yeast after growth in −I media and exposure to 10 and 15% ethanol.

FIG. 2 shows cell viabilities of wild type and opi1 yeast after growth in +I media and exposure to 10, 15, and 18% ethanol concentrations.

FIG. 3 shows the optical density (OD) of wild type and opi1 yeast grown in +I media containing 2 or 12% glucose and 0% ethanol.

FIG. 4 shows the optical density (OD) of wild type and opi1 yeast grown in +I media containing 2 or 12% glucose and 5% ethanol.

FIG. 5 shows the optical density (OD) of wild type and opi1 yeast grown in −I media containing either 2 or 12% glucose and 0% ethanol.

FIG. 6 shows the optical density (OD) of wild type and opi1 yeast grown in −I media containing either 2 or 12% glucose and 5% ethanol.

### DETAILED DESCRIPTION OF THE INVENTION

One aspect of the present invention relates to a method of producing organisms tolerant to alcohol that includes selecting a microorganism needing tolerance to alcohol and modifying the selected microorganism under conditions effective to overproduce inositol by the microorganism compared to when the microorganism is not modified, with the modified microorganism being tolerant to alcohol. Alcohol tolerance is defined as the capacity of a microorganism to grow and/or maintain its ability to carry out biosynthetic reactions (e.g., fermentation) in media containing a high alcohol concentration for non-tolerant microorganisms. High alcohol concentration in fermentation media is the alcohol concentration at which a microorganism has a substantial reduction of its normal growth rate, fermentation capacity or loses viability.

The microorganism can be a species of yeast. The yeast may be a Pichia species, a Candida species, a Schizosaccharomyces species, and a Saccharomyces species. Preferably, the yeast is Saccharomyces cerevisiae.

Modification of the microorganism is carried out by inactivation, deletion, or substitution of a selected gene that prevents overproduction of inositol. It is preferable that the selected gene is OPI1. Inactivation is performed by transforming the microorganism with a nucleic acid construct used to prevent gene expression of the selected gene. The construct includes a 5′ DNA promoter sequence, a nucleic acid molecule that causes inhibition of expression of the selected gene inositol biosynthesis, and a 3′ terminator sequence, where the 5′ DNA promoter sequence and the 3′ terminator sequence are operatively coupled to the nucleic acid molecule.

The nucleic acid molecule may include a nucleotide sequence encoding part or the entire selected gene in anti-sense orientation. Further, this may be followed by a nucleotide sequence encoding part or all of the gene in sense orientation.

Further, the modification of the microorganism includes overexpression of at least a gene encoding a protein in the inositol biosynthesis pathway. It is preferable that the microorganism is Saccharomyces cerevisiae with its INO1 gene being overexpressed or constitutively expressed. The microorganism may be combined with inositol-supplemented media. The INO1 gene coding sequence has a nucleotide sequence of SEQ ID NO: 1 as follows:

ATGACAGAAGATAATATTGCTCCAATCACCTCCGTTAAAGTAGTTACC
GACAAGTGCACGTACAAGGACAACGAGCTGCTCACCAAGTACAGCTAC
GAAAATGCTGTAGTTACGAAGACAGCTAGTGGCCGCTTCGATGTAACG
CCCACTGTTCAAGACTACGTGTTCAAACTTGACTTGAAAAAGCCGGAA
AAACTAGGAATTATGCTCATTGGGTTAGGTGGCAACAATGGCTCCACT
TTAGTGGCCTCGGTATTGGCGAATAAGCACAATGTGGAGTTTCAAACT
AAGGAAGGCGTTAAGCAACCAAACTACTTCGGCTCCATGACTCAATGT
TCTACCTTGAAACTGGGTATCGATGCGGAGGGGAATGACGTTTATGCT
CCTTTTAACTCTCTGTTGCCCATGGTTAGCCCAAACGACTTTGTCGTC
TCTGGTTGGGACATCAATAACGCAGATCTATACGAAGCTATGCAGAGA
AGTCAAGTTCTCGAATATGATCTGCAACAACGCTTGAAGGCGAAGATG
TCCTTGGTGAAGCCTCTTCCTTCCATTTACTACCCTGATTTCATTGCA
GCTAATCAAGATGAGAGAGCCAATAACTGCATCAATTTGGATGAAAAA
GGCAACGTAACCACGAGGGGTAAGTGGACCCATCTGCAACGCATCAGA
CGCGATATCCAGAATTTCAAAGAAGAAAACGCCCTTGATAAAGTAATC
GTTCTTTGGACTGCAAATACTGAGAGGTACGTAGAAGTATCTCCTGGT
GTTAATGACACCATGGAAAACCTCTTGCAGTCTATTAAGAATGACCAT
GAAGAGATTGCTCCTTCCACGATCTTTGCAGCAGCATCTATCTTGGAA
GGTGTCCCCTATATTAATGGTTCACCGCAGAATACTTTTGTTCCCGGC
TTGGTTCAGCTGGCTGAGCATGAGGGTACATTCATTGCGGGAGACGAT
CTCAAGTCGGGACAAACCAAGTTGAAGTCTGTTCTGGCCCAGTTCTTA
GTGGATGCAGGTATTAAACCGGTCTCCATTGCATCCTATAACCATTTA
GGCAATAATGACGGTTATAACTTATCTGCTCCAAAACAATTTAGGTCT
AAGGAGATTTCCAAAAGTTCTGTCATAGATGACATCATCGCGTCTAAT
GATATCTTGTACAATGATAAACTGGGTAAAAAAGTTGACCACTGCATT
GTCATCAAATATATGAAGCCCGTCGGGGACTCAAAAGTGGCAATGGAC
GAGTATTACAGTGAGTTGATGTTAGGTGGCCATAACCGGATTTCCATT
CACAATGTTTGCGAAGATTCTTTACTGGCTACGCCCTTGATCATCGAT
CTTTTAGTCATGACTGAGTTTTGTACAAGAGTGTCCTATAAGAAGGTG
GACCCAGTTAAAGAAGATGCTGGCAAATTCGAGAACTTTTATCCAGTT
TTAACCTTCTTGAGTTACTGGTTAAAAGCTCCATTAACAAGACCAGGA
TTTCACCCGGTGAATGGCTTAAACAAGCAAAGAACCGCCTTAGAAAAT
TTTTTAAGATTGTTGATTGGATTGCCTTCTCAAAACGAACTAAGATTC
GAAGAGAGATTGTTGTAA.

This nucleotide sequence encodes a protein with the amino acid sequence of SEQ ID NO: 2 as follows:

MTEDNIAPITSVKVVTDKCTYKDNELLTKYSYENAVVTKTASGRFDVT
PTVQDYVFKLDLKKPEKLGIMLIGLGGNNGSTLVASVLANKHNVEFQT
KEGVKQPNYFGSMTQCSTLKLGIDAEGNDVYAPFNSLLPMVSPNDFVV
SGWDINNADLYEAMQRSQVLEYDLQQRLKAKMSLVKPLPSIYYPDFIA
ANQDERANNCINLDEKGNVTTRGKWTHLQRIRRDIQNFKEENALDKVI
VLWTANTERYVEVSPGVNDTMENLLQSIKNDHEEIAPSTIFAAASILE
GVPYINGSPQNTFVPGLVQLAEHEGTFIAGDDLKSGQTKLKSVLAQFL
VDAGIKPVSIASYNHLGNNDGYNLSAPKQFRSKEISKSSVIDDIIASN
DILYNDKLGKKVDHCIVIKYMKPVGDSKVAMDEYYSELMLGGHNRISI
HNVCEDSLLATPLIIDLLVMTEFCTRVSYKKVDPVKEDAGKFENFYPV
LTFLSYWLKAPLTRPGFHPVNGLNKQRTALENFLRLLIGLPSQNELRF
EERLL.

The modified microorganism may be tolerant to high osmotic shock.

The present invention is preferably carried out by treating microorganisms in accordance with the disclosure of U.S. Pat. Nos. 6,645,767 and 7,129,079 to Villa et al. which are hereby incorporated by reference in their entirety. Preparation of such microorganisms is summarized in the following paragraphs.

Host haploid yeast strains are first constructed to contain one or more gene mutations which are non-lethal to the host and which can be selected using methods known in the art. Preferably, the gene mutations are in one or more genes of the amino acid biosynthetic pathways of the host which cause an auxotrophic phenotype, such as, for example, his3, leu2, lys1, met15, and trp1 or one or more genes of the nucleotide biosynthetic pathways of the host which cause an auxotrophic phenotype, such as, for example, ade2 and ura3. The gene mutation in the host yeast that causes an auxotrophic phenotype can be a point mutation, a partial or complete gene deletion, or an addition or substitution of nucleotides. These types of mutations cause the strains to become auxotrophic mutants which, in contrast to the prototrophic wild-type strains, are incapable of optimum growth in media without supplementation with one or more nutrients. The mutated genes in the host strain can then serve as auxotrophic gene markers which later can be targets for the insertion of yeast integration plasmids. A targeting gene marker carried on a yeast integration plasmid directs precise insertion of the plasmid into a specific homologous locus in the host cell genome, also called the target gene mutation. Such integration rescues the auxotrophy caused by the target gene mutation in the host haploid cell.

The construction of mutated host yeast strains is carried out by genetic crosses, sporulation of the resulting diploids, tetrad dissection of the haploid spores containing the desired auxotrophic markers, and colony purification of such haploid host yeasts in the appropriate selection medium. All of these methods are standard yeast genetic methods known to those in the art. See, for example, Sherman et al., Methods Yeast Genetics, Cold Spring Harbor Laboratory, NY (1978) and Guthrie et al. (Eds.) Guide To Yeast Genetics and Molecular Biology Vol. 194, Academic Press, San Diego (1991), which are hereby incorporated by reference in their entirety.

The Saccharomyces cerevisiae can be genetically engineered to contain a complete deletion of the open reading frame of the OPI1 gene that prevents the expression of that gene, which is a negative regulator of phospholipid biosynthesis. See White et al. “The OPI1 Gene of Saccharomyces cerevisiae, a Negative Regulator of Phospholipid Biosynthesis, Encodes a Protein Containing Polyglutamine Tracts and a Leucine Zipper,” J Biol Chem 266(2):863-872 (1991) and U.S. Pat. Nos. 5,529,912 and 5,599,701 to Henry et al., which are hereby incorporated by reference in their entirety, for details of construction of opi⁻ strains. The opi⁻ host yeast is then modified to have one or more auxotrophies that can be rescued by transformation with yeast integration plasmids which contain the functional genes homologous to those that are mutated in the host yeast cell. For example, a host yeast cell with a mutated his3 gene which results in a histidine auxotrophy can be complemented by a yeast integration plasmid containing a targeting gene marker which is a functional HIS3 gene. A host yeast cell with, for example, additional mutated genes, such as for example, ade2, leu2, lys1, met15, trp1, ura3, and others, result in auxotrophies which can be rescued by yeast integration plasmids containing as targeting gene markers the functional homologous versions of those genes. In such a case, each yeast integration plasmid of the suite additionally carries a gene of interest, such as an extra copy of the INO1 gene, or any other desired gene.

Yeast integration plasmids of the invention are comprised of the following DNA sequences operably joined together: a selection gene marker; a targeting gene marker; a gene of interest; and a microorganism autonomous DNA start site sequence hereinafter referred to as an origin of DNA replication.

As used herein, a plasmid is an autonomously replicating extrachromosomal DNA, usually circular in shape. Plasmids can be a variety of sizes depending on the genes comprising the integration plasmids. Plasmids present in host microorganisms can carry genes encoding traits which may or may not be present on the microorganism's chromosome. Plasmids can be present in a microorganism in single or multiple copies as separate autonomously replicating units of DNA or can be integrated into a host cell's chromosome.

The sequences for the targeting gene marker, the selection gene marker, the gene of interest and the origin of replication are operably joined together and may be joined together to form yeast integration plasmids with few or no additional plasmid sequences. Alternatively, these DNA sequences can be combined with additional plasmid DNA sequences such as an additional identification sequence that can serve the purpose of identifying/fingerprinting the genetically modified organism by using polymerase chain reaction (“PCR”) procedures. For example, a known non-coding identification sequence can be additionally carried by yeast that can be amplified, for example by PCR procedures, to identify the genetically modified organism such as the modified yeast strains of the present invention. In addition, if desired, integration plasmids may contain a DNA sequence that can be engineered to be recognized by multiple restriction enzymes thereby constituting a multiple cloning site (“MCS”). The DNA sequences can be joined together in any order, as long as they remain operable, and if combined with additional plasmid DNA sequences, the targeting gene marker, the selection gene marker, the gene of interest and the origin of replication sequences can also be separated from each other by other DNA sequences. The DNA sequences can be joined together using standard recombinant DNA methods such as those described by Sambrook et al., Molecular Cloning: A Laboratory Manual, 2^{nd} Ed., Cold Spring Harbor Laboratory, Cold Spring Harbor, N.Y. (1989), which is hereby incorporated by reference in its entirety.

The selection gene marker allows for replication of the yeast integration plasmid in a host plasmid amplification microorganism such as bacteria or yeast and also allows for selection of the transformed host colonies containing the integration plasmids to be amplified. A gene used as a selection gene marker is preferably a yeast gene, and more preferably is a yeast gene that complements a selectable auxotrophy in a plasmid amplification host microorganism used for replication of integration plasmids. A yeast gene such as LEU2 of S. cerevisiae is preferred as a selection gene marker in the present invention, because it is able to rescue the leucine auxotrophy of both a specific bacterial replication host, and in certain cases discussed herein, that of a host yeast which contains a mutated leu2 gene. A selection gene marker such as the LEU2 gene carried on yeast integration plasmids of the invention replaces traditional bacterial drug resistance gene markers such as amp^{r}.

A targeting gene marker carried on a yeast integration plasmid directs its stable integration to its specific homologous locus in the host strain which preferably contains a natural or engineered target gene mutation, i.e., a point mutation, a partial gene deletion, or total gene deletion. For example, a host yeast strain that carries a his3 mutated gene is complemented by the functional HIS3 targeting gene marker provided by a yeast integration plasmid resulting in the release of the host auxotrophy upon integration of the plasmid. A targeting gene marker may also additionally function as a selection gene marker for DNA plasmid amplification in bacterial or yeast plasmid amplification hosts, provided the gene is able to rescue or complement an auxotrophy of the bacterial or yeast amplification host. For example, the plasmid pVG102-A containing the LEU2 gene can be amplified in the E. coli JA221 bacterial host (ATCC Deposit No. 33875) which is auxotrophic for leucine. In such a case LEU2 is the selection gene marker for the plasmid amplification step. After amplification and purification, if the pVG102-A plasmid is used to transform a yeast host that is auxotrophic for leucine, then LEU2 would serve the additional purpose of a targeting gene marker. Similarly, if one uses an E. coli MH1066 bacterial host (see, Hall et al., “Targeting of E. coli Beta-galactosidase to the Nucleus in Yeast,” Cell 36:1057 (1984), which is hereby incorporated by reference in its entirety) which is auxotrophic for leucine, tryptophan, and uracil, the LEU2, TRP1, and URA3 genes in each of the integration plasmids could also serve a dual purpose of selection gene marker and targeting gene marker in the transformation of yeast hosts with auxotrophies in any or all of these genes.

The gene of interest in the yeast integration plasmids of the invention that is desired to be expressed in the host yeast can be homologous or heterologous to the host yeast genome. In the case where the production of inositol and its metabolites are desired, the gene of interest is INO1. The gene of interest can be the same gene in each member of a suite of yeast integration plasmids such as the INO1 gene in the exemplified embodiment of the present invention, or the gene of interest can be a different gene for each member of the suite of plasmids that is desired to be expressed in yeast. This feature of the invention allows the engineering of genetically modified yeast or other hosts with either multiple copies of the same gene of interest, causing overproduction of the encoded protein, or allows the genetic engineering of new metabolic pathways or the modification of existing metabolic pathways in the chosen host. For instance, one can engineer a new pathway to produce a given metabolite that the host yeast does not produce naturally by inserting the appropriate genes to create such a novel metabolic pathway which in turn produces the metabolite. It is to be understood that it is also possible to construct yeast integration plasmids to contain more than one gene of interest in a tandem repeat configuration such that each copy of the gene of interest is in the same head to tail orientation.

Yeast integration plasmids are amplified episomally in a host microorganism such as bacteria or yeast in order to have enough plasmid DNA to perform the subsequent host yeast transformation and integration steps. Preferably, the yeast integration plasmids of the present invention are replicated in bacteria because of ease of purification as compared to plasmids amplified in yeast. As stated above, in such cases the yeast integration plasmids preferably contain a bacterial origin of replication such as ORI derived from the plasmid pUC18 although other bacterial origins of replication can be used. However, as stated above, in cases where it is desired to replicate yeast integration plasmids in yeast, an origin of replication for yeast can be carried on yeast integration plasmids to allow for autonomous replication of the plasmid in a yeast plasmid amplification host so long as it is removed from the amplified plasmids.

Once the auxotrophic host haploid strains of both mating types are constructed and the yeast integration plasmids are also constructed, amplified, and purified, the next step is the sequential transformation of each mating type haploid with the appropriate integration plasmid that can complement the host auxotrophy or auxotrophies. It is known that one of the most stable ways to introduce and maintain a gene of interest into a host cell is by integration of the gene by homologous recombination. Homologous recombination in the present invention consists of the insertion of an entire yeast integration plasmid, directed by its targeting gene marker, into a specific mutated target locus in the host genome, a target gene mutation, which is a mutated gene that causes an auxotrophy in the host. The target gene mutation at the target locus in the host cell and the targeting gene marker in the yeast integration plasmid are said to be homologous. For instance, a his3 mutant gene at the target locus in the host cell genome is homologous to a functional HIS3 targeting gene marker carried on an integration plasmid. Once the recombination occurs, the targeting gene marker and all other genes carried by the integration plasmid, including the gene of interest, are stably integrated into the host genome. Therefore, in haploid host cells a single copy of a gene of interest can be integrated into a specific target locus in the host genome. In order to transform the host yeast, an integration plasmid is first linearized by opening the plasmid with restriction enzymes preferably at a given restriction site within the targeting gene marker. The linearized plasmid is then transformed into the host cell and finally is successfully homologously recombined with the target locus.

Yeast strains are transformed with isolated plasmid DNA using the lithium acetate method described by Ito et al., “Transformation of Intact Yeast Cells Treated with Alkali Cations,” J Bacteriol 153:163-168 (1983) as modified by Hirsch et al., “Expression of the Saccharomyces cerevisiae Inositol-1-phosphate Synthase (INO1) Gene is Regulated by Factors that Affect Phospholipid Synthesis,” Mol Cell Biol 6:3320-3328 (1986), which are hereby incorporated by reference in their entirety. Yeast strains may also be transformed by the methods described by Guthrie et al. (Eds.) Guide To Yeast Genetics and Molecular Biology, Vol. 194, Academic Press, San Diego (1991), which is hereby incorporated by reference in its entirety. Where indicated, directed transformations and linearized plasmid transformations are performed by digesting plasmids at specific endonuclease restriction sites.

After the parent haploids are transformed with the appropriate integration plasmids and colony purified in selective medium, the transformed host haploids of opposite mating types are crossed to produce prototrophic diploids that contain multiple copies of the gene of interest at precise loci in the parent host cell genome but which lack any drug resistance gene markers. The diploid host strain carries at least one copy from each haploid mating type of a single gene of interest or a set of different genes of interest that completes a homologous metabolic pathway or constitutes a new heterologous metabolic pathway. Furthermore, if a haploid auxotrophic strain with only one mutated gene acquires a functional copy of its homologous gene from an integration plasmid, the strain will become prototrophic and will grow in synthetic minimal media without additional nutritional supplementation. In addition, when haploid strains of opposite mating types, each containing different auxotrophies but complementary to one another, are crossed, the resulting diploid becomes prototrophic and able to grow in minimal growth media. That is, the functional gene copy of a haploid strain of a mating type complements the gene mutation of the opposite mating type. For industrial applications, it is preferable to have at least diploid strains that are completely prototrophic.

The use of several different target loci in the host cell genome in the present invention may be used to increase the genetic stability of the host cells which are transformed with integration plasmids of the present invention. The insertion of the yeast integration plasmids carrying genes of interest into different target loci prevents the spontaneous recircularization and excision of integration plasmids which could take place when all the integration plasmids are inserted in a single target locus in the host genome.

According to a related aspect, the present invention also relates to a method of producing alcohol that includes providing a microorganism that is tolerant to alcohol and modified to increase production of inositol by the microorganism compared to when the microorganism is not modified. A fermentable feedstock is treated with the modified microorganism under conditions effective to produce alcohol. This aspect of the present invention should be carried out with the modified microorganisms as described above.

The fermentable feedstock may be a cellulosic material and be selected from the group consisting of corn, trees, grasses, hemp, and sugarcane. The fermentable feedstock may be supplemented with inositol.

Fermentable feedstocks can be in the form of biomass, such as cellulose, hemicellulose, lignin, protein and carbohydrates such as starch and sugar. Common forms of biomass include trees, shrubs and grasses, corn, and corn husks, as well as municipal solid waste, waste paper, and yard waste. Biomass high in starch, sugar or protein, such as corn, grains, fruits and vegetables, are usually consumed as food. Conversely, biomass high in cellulose, hemicellulose, and lignin are not readily digestible and are primarily utilized for wood and paper products, fuel, or are disposed of Ethanol and other chemical fermentation products typically have been produced from sugars derived from feedstocks high in starches and sugars, such as corn.

Agricultural biomass includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, vines and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination or mixture thereof.

Biomass includes virgin biomass and/or non-virgin biomass such as agricultural biomass, commercial organics, construction and demolition debris, lignocellulose, municipal solid waste, waste paper, and yard waste. The present invention relates to crushed or broken down plant material.

Fermentation materials include any material or organism capable of producing a fermentation product (e.g., alcohol, particularly C₁ to C₆ alcohols, and, more particularly, ethanol). Ethanol includes ethyl alcohol or mixtures of ethyl alcohol and water. In general, fermentation is a process carried by bacteria, such as Zymomonas mobilis and Escherichia coli; yeast, such as Saccharomyces cerevisiae or Pichia stipitis; and fungi that are natural ethanol-producers. Alternatively, fermentation can be carried out with engineered organisms that are induced to produce ethanol through the introduction of foreign genetic material (such as pyruvate decarboxylase and/or alcohol dehydrogenase genes from a natural ethanol producer). Further, mutants and derivatives, such as those produced by known genetic and/or recombinant techniques, of ethanol-producing organisms, which mutants and derivatives have been produced and/or selected on the basis of enhanced and/or altered ethanol production.

Ethanol fermentation is the biological process by which sugars such as glucose, fructose, and sucrose are converted into cellular energy and thereby producing ethanol and carbon dioxide as metabolic waste products. Yeast carry out ethanol fermentation on sugars in the absence of oxygen. Because the process does not require oxygen, ethanol fermentation is classified as anaerobic. Ethanol fermentation is responsible for the rising of bread dough, the production of ethanol in alcoholic beverages, and for much of the production of ethanol for use as fuel.

The conversion of sugar into ethanol by yeast fermentation is well known, and many sugar-containing materials have been investigated for use in this method of production of ethanol. In general, these processes are based on the initial production of a sugar-containing liquid, followed by liquid-phase yeast fermentation thereof. Sugar beets are a well-known and widely-used source of sugars, particularly sucrose. See U.S. Pat. No. 4,490,469 to Kirby et al. and Atiyeh et al., “Production of Fructose and Ethanol from Sugar Beet Molasses Using Saccharomyces cerevisiae ATCC 36858,” Biotechnol Prog 18(2):234-239 (2002), which are hereby incorporated by reference in their entirety.

### EXAMPLES

The following examples are provided to illustrate embodiments of the present invention but are by no means intended to limit its scope.

### Materials and Methods for Examples 1-2

### Strains

S. cerevisiae wild type diploid MVY#2008 in the W303 background and its mutant derivative MVY#2015 (opi1) were used in all plating assays. S. cerevisiae wild type haploid MVY#19 in the S288C background and its mutant derivative MVY#2013 (opi1) were used in all growth curve assays. The genotypes of these strains are presented in Table 1.

TABLE 1
S. cerevisiae Strains Used

| Strain | Genotype | Source |
|---|---|---|
| AID | ade1/ade1 ino1/ino1 MATa/α | S. A. Henry |
| MVY#2008 | ade2-1/ADE2 his3-11/HIS3 leu2- | M. Villa |
| | 3/LEU2 lys2/LYS2 trp1-1/TRP1 | |
| | ura3-1/URA3 opi1-1/opi1-1 Mat | |
| | a/Mat alpha | |
| MVY#2015 | ADE2/ADE2 HIS3/HIS3 l LEU2/ | M. Villa |
| | LEU2 LYS2/LYS2 TRP1/TRP1 | |
| | URA3/URA3 OPI1/OPI1 | |
| | Mat a/Mat alpha | |
| MVJY#19 | his3Δ1 leu2Δ0 lys2Δ0 ura3Δ0 Mat α | M. Villa |
| MVJY#13 | his3Δ1 leu2Δ 0 lys2Δ0 ura3Δ0 | M. Villa |
| | opi1Δ::KanMX4 Mat α | |

### Innoculum Preparation

S. cerevisiae strains that had been stored at −80° C. were plated onto YPD plates (1% yeast extract, 2% peptone, 2% glucose and 2% agar). Plates were incubated at 30° C. for 48 hr (Low Temperature Incubator, Fisher Scientific, Pittsburgh, Pa.). One colony from each plate was removed with a sterile loop and patched on a fresh YPD plate, incubated at 30° C. for 48 hr, and stored at 4° C. for further use. Small amounts of the single colony patch were removed from the plate with a sterile loop and suspended in 125-mL polycarbonate Erlenmeyer flasks containing 25-mL YPD (1% yeast extract, 2% peptone, and 2% glucose). The flasks were covered with screw caps and placed in an incubator-shaker (G24 Environmental Shaker, New Brunswick Scientific Company, Inc., Edison, N.J.) at 30° C. and 250 rpm for 12 hr. Optical density (OD) at 600 nm was measured for each culture with a spectrophotometer (8453 UV-Vis Spectroscopy System, Agilent Technologies, Inc., Palo Alto, Calif.). All further discussion of OD will imply measurement at 600 nm. The culture with an OD of 2-3 was selected for use in subsequent experiments.

### Assay for Opi⁻ Phenotype

The Opi⁻ phenotype was tested on both wild type and opi1 strains to ensure the absence/presence of this phenotype throughout all plating and growth curve experiments, as described by, et al. (White et al., “The Gene of Saccharomyces cerevisiae, a Negative Regulator of Phospholipid Biosynthesis, Encodes a Protein Containing Polyglutamine Tracts and a Leucine Zipper,” J Biol Chem 266(2):863-872 (1991), which is hereby incorporated by reference in its entirety). Composition of synthetic complete medium, is also described by White (White et al., “The OPI1 Gene of Saccharomyces cerevisiae, a Negative Regulator of Phospholipid Biosynthesis, Encodes a Protein Containing Polyglutamine Tracts and a leucine Zipper,” J Biol Chem 266(2):863-872 (1991), which is hereby incorporated by reference in its entirety). Inositol-containing medium (which will be referred to as +I) and plates were identical to synthetic complete medium (which will be referred to as −I), except for the inclusion of 75 μM myo-inositol. Small aliquots of each culture were plated onto inositol-free plates and incubated at 30° C. for 48 hr.

A culture of the tester strain, AID (refer to Table 3 for genotype), was prepared by suspending a single colony into a 250-mL Erlenmeyer flask containing 50-mL of YPD medium, and incubating at 30° C. and 250 rpm for 24 hr. The cells were washed twice with sterile water and centrifuged at 4,500 rpm for 5 min. The cell-free supernatant was discarded after each washing. The cell pellet was re-suspended in sterile water and stored at 4° C. Each inositol-free plate patched with the strain to be evaluated was sprayed with the AID tester strain and incubated at 30° C. for 48 hr. The AID strain is diploid homozygous for both the ino1 and ade1 markers. It, therefore, exhibits a red phenotype and can only grow on inositol-free plates when the existing strain produces and excretes inositol into the medium around the patch. In these plates, a red halo indicates the presence of the Opi⁻ phenotype for the strain in question (White et al., “The OPI1 Gene of Saccharomyces cerevisiae, a Negative Regulator of Phospholipid Biosynthesis, Encodes a Protein Containing Polyglutamine Tracts and a leucine Zipper,” J Biol Chem 266(2):863-872 (1991), which is hereby incorporated by reference in its entirety).

### Plating Assays

The method for examining ethanol tolerance using a plating technique was adapted from Chi et al. (Chi et al., “Role of Phosphatidylinositol (PI) in Ethanol Production and Ethanol Tolerance By a High Ethanol Producing Yeast,” J Industrial Microbiology Biotechnology 22:58-63 (1999), which is hereby incorporated by reference in its entirety). Inocula of wild type and opi1 yeast strains were prepared as previously described. Cells were centrifuged at 4,500 rpm for 5 min at 4° C. (Sorvall Legend R T, Kendro Laboratory Products, Asheville, N.C.), and the supernatant was removed. Cells were washed twice with −I media, centrifuged at 4,500 rpm for 5 min, and the resulting pellet was stored at 4° C.

The cell pellets were re-suspended in inositol-free media and the OD was measured. Cells were inoculated into a 500-mL polycarbonate Erlenmeyer flask containing 110-mL of synthetic media either containing (+I) or lacking inositol (−I) at a final OD of 0.1. The cell cultures were placed in an incubator-shaker at 30° C. and 250 rpm until the OD reached 0.5.

Two volumes of cell suspension containing 1×10⁸ cells—previously determined by cell counting with a hemacytometer (Bright-Line Hemacytometer, Reichert Scientific Instruments, Buffalo, N.Y.)—were removed from the flasks and placed in separate sterile 50-mL centrifuge tubes. Cells were centrifuged at 4,500 rpm for 5 min at 4° C. and the supernatant was removed. Cells were washed twice with either −I or +I media as previously described, and the resulting supernatants were discarded. Ten mL of ethanol (10, 15 or 18% (v/v)) was added to one of the 50-mL centrifuge tubes containing a washed cell pellet and this represented the “shocked culture.” As a control, 10-mL of sterile water was added to the second 50-mL tube containing a washed cell pellet. Two 25-μl aliquots of the control culture were removed and plated on YPD plates, representing time zero of the experiment. Cell suspensions were transferred from 50-mL centrifuge tubes to 25-mL sterile glass tubes. The glass tubes were placed in an incubator-shaker at 30° C. and 250 rpm.

Samples of both the shocked and control cultures were taken every hour for 4 hr. Shocked cells were diluted in ethanol (10, 15, or 18%, consistent with the concentration of the shocked culture) at dilution factor of 1:1000. Control cells were diluted in sterile water at the same dilution factor. Duplicate 25-μl aliquots were plated on YPD plates for each sample of either shocked or control cell cultures over the 4 hr time course. All plates were placed in a 30° C. incubator. Colonies were counted on all plates after 48 and 72 hr. Duplicate plate counts were averaged. Ethanol tolerance of each strain, in terms of cell viability, in either +I or −I media over a range of ethanol concentrations, was expressed as a ratio of the number of colonies counted in the shocked culture at 72 hr to the number of colonies counted in the control culture at 72 hr. Cell viability in the control culture at time-zero of the experiment was assumed to be 100%.

Ethanol tolerance of each strain was calculated, in terms of cell viability (CV), using the number of colony forming units (CFUs) in the shocked and control cultures after 72 hr of incubation using the following equation:

$$CV = \frac{{CFU}_{S}}{{CFU}_{N}} * 100 \qquad ( 1 )$$

where

CFU_{S}=average number of CFUs counted in shocked culture

CFU_{N}=average number of CFUs counted in nonshocked culture

Cell viability in the control culture at time-zero of the experiment was assumed to be 100%. All ethanol concentrations are volume-volume percentages (v/v), while all glucose concentrations are in weight-volume percentages (w/v).

### Growth Curve Analysis

The method for examining ethanol tolerance by profiling yeast growth was adapted from You et al. (You et al., “Ethanol Tolerance in the Yeast Saccharomyces cerevisiae is Dependent on Cellular Oleic Acid Content,” Applied Environ Microbiology 69:1499-1503 (2003), which is hereby incorporated by reference in its entirety). Inocula of wildtype and opi1 yeast strains were prepared as previously described (see Plating Assays) except a Marathon 21K/BR bench top centrifuge (Fisher Scientific, Inc., Hampton, N.H.) was used. The cell pellet was suspended in —I media and the OD was measured by pipetting 200-1 μL of the cell suspension into a clean well of a 96 well plate and reading OD in a plate reader (Synergy HT Multidetection Microplate Reader, Bio-Tek Instruments, Inc., Winooski, Vt.).

Cells were inoculated into 125-mL polycarbonate Erlenmeyer flasks with 50-mL of +I or −I containing either 2% or 12% glucose and 0 or 5% ethanol, at a final OD of 0.05. Each media type was performed in triplicate for both yeast strains. Flasks were placed in an incubator-shaker at 30° C. and 250 rpm (Gyratory Waterbath Shaker, New Brunswick Scientific, Edison, N.J.). The OD of each culture was measured over a period of at least 36 hr or until the cell density had stabilized for a period of no less than 12 hr. Triplicate OD measurements were performed for each sample and measurements were averaged.

Along with ODs, ethanol and glucose concentrations were determined for samples taken from both opi1 and wildtype cells grown in −I media containing 12% glucose and 5% ethanol. One 2-mL sample was removed from the flasks periodically, tested for OD, and immediately passed through 0.22-μm filters (Millex-GP Syringe Driven Filter Unit, Millipore Corporation, Billerica, Mass.) to remove cells, thereby stopping any further utilization of substrate, nutrients, and/or products in the supernatant. Samples were stored at −20° C. for further analysis. Glucose and ethanol concentrations were determined by a YSI 2700 SELECT™ Biochemistry Analyzer (Giangarlo Scientific Company, Pittsburgh, Pa.).

### Example 1

### Plating Assays

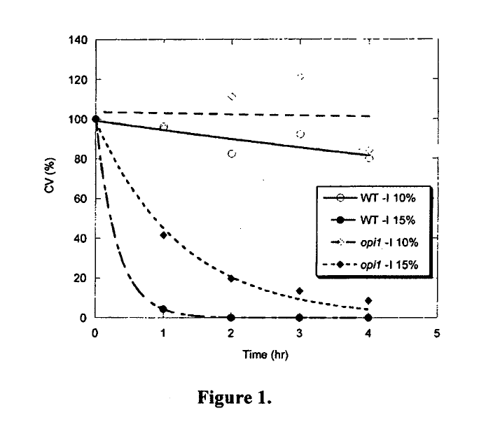

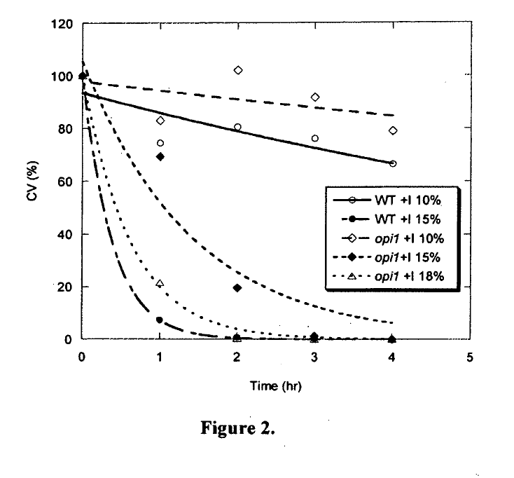

Values for CV are plotted versus time in FIG. 1 for the opi1 and the wild type strains after growth in −I media are. For ethanol exposure of 10 and 15%, the opi1 cells had higher CV values and the rate of decrease in CV with time was less than that observed with wild type cells. In 18% ethanol, CV values of opi1 cells fell to near 0% in the first hour after growth in −I. Similar results were obtained for the opi1 and the wild type strains after growth in +I media are (see FIG. 2). In addition, for I+, opi1 showed higher tolerance to 18% ethanol than the wild type did to 15% ethanol after growth in +I.

To model the data presented in FIGS. 1 and 2 and to estimate specific death rates a simple first-order kinetic model was fitted to the data using the nonlinear parameter estimation tool in KaleidaGraph (Synergy Software, Reading, Pa.):

y=a*e^{-dt} (2)

where

a=initial percent cell viability at time zero

d=specific death rate, (hr⁻¹)

The resulting curve fits obtained for this model and the experimental data are shown in FIGS. 1 and 2, and the estimates of the d for all media types and strains are summarized in Table 2.

TABLE 2
Specific Death Rates of Wild Type and opi1 Yeast Cultures Shocked
in 10, 15, or 18% Ethanol

Specific Death Rates in 2%
Glucose (d, hr⁻¹)

| Media Type | WT | R² | opi1 | R² |
|---|---|---|---|---|
| I+ | | | | |
| 10% Ethanol | 0.084 | 0.70 | 0.036 | 0.26 |
| 15% Ethanol | 2.61 | 1 | 0.71 | 0.93 |
| 18% Ethanol | NA | NA | 1.61 | 1 |
| I− | | | | |
| 10% Ethanol | 0.049 | 0.65 | 0.006 | 0.005 |
| 15% Ethanol | 3.14 | 1 | 0.79 | 0.99 |
| 18% Ethanol | NA | NA | NA | NA |

Cells marked “NA” indicated specific death rates that could not be determined by this method.

Estimates for d and the plots in FIGS. 1 and 2 clearly show the effects of ethanol on yeast cell viability. In 10% ethanol, d values were less than 0.1 hr⁻¹ for all strains, regardless of whether inositol was present in the growth media or not. While the R² values reported in Table 2 for the 10% ethanol curve fits are very low, most likely due to the use of an exponential model when a linear model would be more appropriate, the d values predicted by this model are consistent with a general trend of declining CV values after exposure to 10% ethanol. In 15 and 18% ethanol, estimated d values were lower for cells grown in the presence of inositol, +I, than those grown without inositol, −I, indicating that inositol supplementation in wild type and opi1 strains increased ethanol tolerance.

Cells carrying the opi1 mutation had a clear advantage over the wild type cells when exposed to 15% ethanol after growth in −I media. The value for d of wild type cells was nearly four times that of opi1 cells experiencing the same growth conditions and ethanol treatment (3.14 hr⁻¹ for wild type and 0.79 hr⁻¹ for opi1). The difference in d values for the two strains after growth in +I media and exposure to 15% ethanol was nearly as sizeable—wild type cells died at a rate just over 3.5 times that of opi1 cells (2.61 hr⁻¹ for wild type and 0.71 hr⁻¹ for opi1). It is interesting that the only incidence of significant survival in 18% ethanol occurred in opi1 cells after growth in +I media. In fact, d was 38% lower than that of wild type cells grown in +I media and exposed to 15% ethanol (1.61 hr⁻¹ in opi1 in 18% ethanol and 2.61 hr⁻¹ for wild type in 15% ethanol).

Of particular interest is the small variability in d values of opi1 cells grown in +I and −I media. During exposure to 15% ethanol, the d estimated for opi1 increased 11% after growth in −I media in comparison to +I media (0.79 hr⁻¹ after growth in −I, 0.71 hr⁻¹ for +I). For the wild type strain, however, the d value after growth in −I media was 20% higher than the d value after growth in +I (2.61 hr⁻¹ after growth in −I, 3.14 hr⁻¹ for +I).

The main distinction between these two strains of yeast is the inherent ability of opi1 to produce inositol constitutively, due to the lack of negative regulation of the INO1 gene. This trait allows the opi1 mutant to maintain a higher level of both intracellular inositol and plasma membrane PI content (Jiranek et al., “Pleiotropic Effects of the opi1 Regulatory Mutation of Yeast: its Effects on Growth and on Phospholipid and Inisitol Metabolism,” Microbiology 144:2739-2748 (1998), which is hereby incorporated by reference in its entirety). Assuming that this is the principal difference between the wild type and opi1 strains used in this study, one can infer that this alteration in inositol and PI content is responsible for the varied response to ethanol as demonstrated here. Furthermore, the results of the plating assays presented here support conclusions of two other reports on the influence of inositol on the ethanol tolerance of yeast: supplying inositol to yeast cells increases cell viability in the presence of added ethanol (Chi et al., “Role of Phosphatidylinositol (PI) in Ethanol Production and Ethanol Tolerance By a High Ethanol Producing Yeast,” J Industrial Microbiology and Biotechnology 22:58-63 (1999); and Furukawa et al., “Effect of Cellular Inositol Content on Ethanol Tolerance of Saccharomyces cerevisiae in Sake Brewing,” J Bioscience Bioengineering 98(2):107-113 (2004), which are hereby incorporated by reference in their entirety).

### Example 2

### Growth Curve Assays

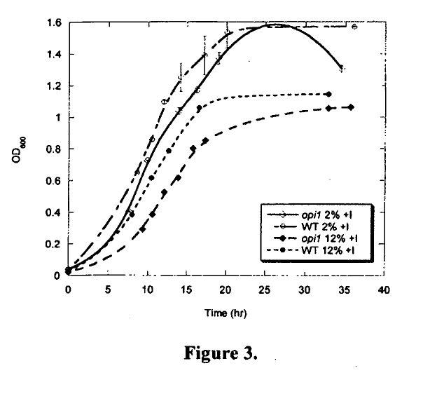

Cell growth, using OD as a surrogate for cell mass, versus time is plot in FIGS. 3-6 for wild type and opi1 in +I and −I media containing 2% or 12% glucose and 0% or 5% ethanol. Error bars are also plotted for all data, representing the standard deviation of the triplicate measurements at each time point. A lower ethanol concentration than was used in the plating assays was employed because of the increased sensitivity of cell growth to ethanol and glucose (D'Amore et al., “A Study of Ethanol Tolerance in Yeast,” Crit Rev Biotechnology 9(4):287-304 (1990), which is hereby incorporated by reference in its entirety). Previous studies employing this method of evaluating ethanol tolerance used media containing various concentrations of glucose: 2% (You et al., “Ethanol Tolerance in the Yeast Saccharomyces cerevisiae is Dependent on Cellular Oleic Acid Content,” Applied Environ Microbiology 69:1499-1503 (2003), which is hereby incorporated by reference in its entirety), 5% (Aguilera et al., “Relationship between Ethanol Tolerance, H+-ATPase Activity and the Lipid Composition of the Plasma Membrane in Different Wine Yeast Strains,” Int J Food Microbiology 110:34-42 (2006), which is hereby incorporated by reference in its entirety), and 20% (Beaven et al., “Production and Tolerance of Ethanol in Relation to Phospholipid Fatty Acyl Composition in Saccharomyces cerevisiae NCYC 431,” J Gen Microbiology 128:1447-1455 (1982), which is hereby incorporated by reference in its entirety). Osmotic pressure due to high glucose concentrations (generally above 5%) has been shown to inhibit growth rate (Casey et al., “Ethanol Tolerance in Yeasts,” CRC Crit Rev Microbiology 13(3):219-280 (1986), which is hereby incorporated by reference in its entirety). However, industrial ethanol production requires high glucose feedstocks in order to minimize energy requirements for distillation of fermentation products (Jacques et al., The Alcohol Textbook, 3^{rd} ed. Nottingham Press, Nottingham, United Kingdom, (1999), which is hereby incorporated by reference in its entirety). In this study, growth curve assays for both strains were performed in a low glucose concentration (2%) to ensure the absence of osmotic effects from glucose, and a high glucose concentration (12%) to more accurately portray the conditions in an industrial process.

The differences in growth between wild type and opi1 yeasts in growth media without ethanol is illustrated in FIG. 3. In +I, with 2% and 12% glucose concentrations, the wild type growth appears to surpass that of opi1, if only slightly. Reduced lag times and higher final cell concentrations were also observed for wild type cells in 12% glucose (see FIG. 3). The enhanced growth of wild type cells in +I is possibly due to the de novo synthesis of inositol in opi1 cells, requiring energy that could otherwise be used for cellular growth.

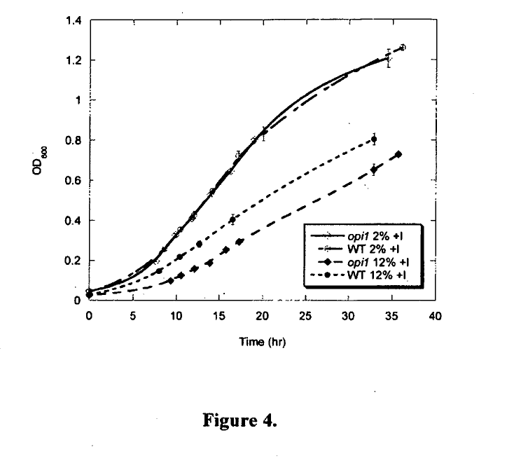

The differences in growth between wild type and opi1 yeasts in growth media containing 5% ethanol in +I media are shown in FIG. 4. Wild type and opi1 had very similar growth curves in 2% glucose. In 12% glucose, the two strains appear to grow at a similar growth rate, but wild type cells had a shorter lag phase.

It is interesting to note that while wild type had a slight advantage over opi1 in +I media containing 2% glucose without ethanol, the growth rates appear to be virtually equal when ethanol was present. This difference in the comparative profiles of the two yeast strains exposes a distinction in the opi1 strain with regard to inositol production. This distinction is best investigated by performing experiments in −I media. The opi1 strain has been shown to accumulate higher levels of PI in the cellular membrane in −I media than the wild type yeast (Jiranek et al., “Pleiotropic Effects of the opi1 Regulatory Mutation of Yeast: its Effects on Growth and on Phospholipid and Inisitol Metabolism,” Microbiology 144:2739-2748 (1998), which is hereby incorporated by reference in its entirety). If PI is indeed responsible for increased ethanol tolerance, opi1 cells should have an advantage over wild type cells when grown in the presence of ethanol.

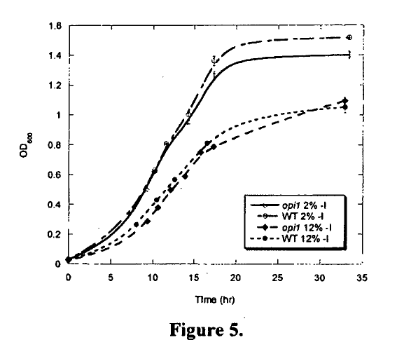

The growth curves for wild type and opi1 for growth in −I without ethanol can be seen in FIG. 5. In 2% and 12% glucose, the growth profiles of the two strains appear to be virtually equal. It has been shown that the wild type and opi1 yeasts show similar growth trends in −I media containing 2% glucose (Jiranek et al., “Pleiotropic Effects of the opi1 Regulatory Mutation of Yeast: its Effects on Growth and on Phospholipid and Inisitol Metabolism,” Microbiology 144:2739-2748 (1998), which is hereby incorporated by reference in its entirety). Therefore, these results are consistent with the literature. Addition of ethanol in −

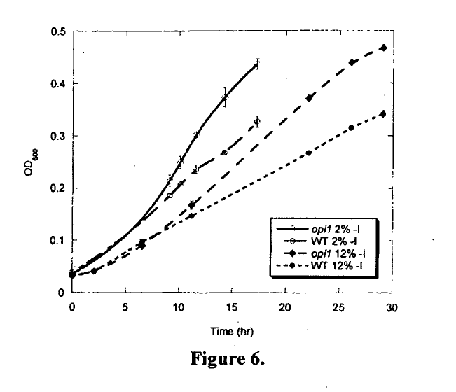

I media, as seen in FIG. 6, created an environment in which the opi1 strain had a clear advantage over the wild type strain in both 2% and 12% glucose concentrations. While the lag phases of these strains are similar in both 2% and 12% glucose, growth rates and final cell concentration of the opi1 mutant exceeded those of the wild type.

The difficulty in discerning differences in lag phase, growth rate, and the extent of growth from simply looking at these plots is obvious from the preceding discussion. Specific growth rate, or the growth rate over time per unit of biomass, was calculated in order to provide a quantitative assessment of the impact of experimental conditions on growth rate. Specific growth rates were estimated for both yeast strains for all of the conditions mentioned using the following first order kinetic equation:

y=a*e^{μt} (3)

where

a=initial cell concentration, in terms of OD

μ=specific growth rate, (hr⁻¹)

This equation was fit to the data presented in FIGS. 3 through 6 using the nonlinear parameter estimation tool in KaleidaGraph. The estimated values for μ for both strains in all growth conditions are summarized in Table 3 below.

TABLE 3
Specific Growth Rates of Wild Type and opi1 Yeast Cultures Grown
in Either −I or +I Media Containing 2% (a) or 12% (b) Glucose
and 0% or 5% Ethanol Concentrations (v/v).
a)

| | Specific Growth Rates in 2% | | | |
|---|---|---|---|---|
| | Glucose (μ, hr⁻¹) | | | |
| Media Type | WT | R² | opi1 | R² |
| I+ | | | | |
| 0% Ethanol | 0.153 ± 0.001 | 1 | 0.125 ± 0.036 | 0.934 |
| 5% Ethanol | 0.111 ± 0.007 | 0.988 | 0.118 ± 0.012 | 0.973 |
| I− | | | | |
| 0% Ethanol | 0.102 ± 0.008 | 0.988 | 0.100 ± 0.010 | 0.973 |
| 5% Ethanol | 0.065 ± 0.004 | 0.988 | 0.102 ± 0.011 | 0.976 |

b)

| | Specific Growth Rates in 12% | | | |
|---|---|---|---|---|
| | Glucose (μ, hr⁻¹) | | | |
| Media Type | WT | R² | opi1 | R² |
| I+ | | | | |
| 0% Ethanol | 0.102 ± 0.016 | 0.959 | 0.142 ± 0.012 | 0.981 |
| 5% Ethanol | 0.109 ± 0.010 | 0.986 | 0.127 ± 0.007 | 0.993 |
| I− | | | | |
| 0% Ethanol | 0.100 ± 0.007 | 0.995 | 0.102 ± 0.013 | 0.954 |
| 5% Ethanol | 0.057 ± 0.007 | 0.964 | 0.076 ± 0.010 | 0.975 |

It is typical for yeast to experience a lag phase, during which there is no observable increase in cell concentration, when inoculated into fresh medium. In light of the fact that lag phase is dependent on many variables, and that lag phase in response to a given condition is not consistent among all strains of yeast, it is very difficult to model. While all cultures in this study were of similar age, multiple types of growth media were used and each strain responded differently to each media type. Hence, lag phase data has been omitted for this analysis and only exponential growth data has been used for the purpose of quantitative comparison of the wild type and opi1 strains.

The quantitative analysis of the μ values of the wild type and opi1 strains in all growth conditions is, in general, in agreement with the qualitative comparison of the complete growth profiles discussed above. As seen in FIGS. 3 through 6, both strains can have similar growth rates during the phase of exponential growth, but exhibit different length of lag phase and extent of fermentation, resulting in different final cell concentrations. Lag phase reflects the cells ability to adapt to new media. Moreover, since lag phase and extent of fermentation are not included in this model, the specific growth rates here do not completely describe the cells ability to deal with the effects of ethanol. While R² values for these curve fits were all above 0.90, a minimal number of data points in the exponential growth phase for some of the cultures resulted in μ values that did not necessarily agree with the qualitative assessment of the above plots.

A very indicative trend seen in Table 3 is that in 5% ethanol μ values of opi1 always exceeded those of the wild type, regardless of inositol or glucose concentration whereas growth rates in the same medium without ethanol were virtually equal. Although the increase in μ value in 5% ethanol for opi1 was small in +I media (5% higher in 2% glucose and 16% higher in 12% glucose), it was quite significant in −I media (57% higher in 2% glucose and 33% higher in 12% glucose).

These results indicate that opi1, with the ability to constitutively produce inositol regardless of media composition, has a marked advantage over the wild type yeast in environments where ethanol is present and inositol is not. As previously discussed, it is most likely the composition of the cellular membrane that imparts this advantage for the opi1 yeast. The opi1 strain has been shown to accumulate higher levels of PI in the cellular membrane without media supplementation of inositol (Jiranek et al., “Pleiotropic Effects of the opi1 Regulatory Mutation of Yeast: its Effects on Growth and on Phospho lipid and Inisitol Metabolism,” Microbiology 144:2739-2748 (1998), which is hereby incorporated by reference in its entirety). Furukawa et al. (Furukawa et al., “Effect of Cellular Inositol Content on Ethanol Tolerance of Saccharomyces cerevisiae in Sake Brewing,” J Bioscience Bioengineering 98(2):107-113 (2004), which is hereby incorporated by reference in its entirety) showed that in the presence of ethanol, ATPase activity was inhibited to a lesser extent in cells having a higher proportion of inositol-containing phospholipids. The functionality of many cellular transport systems is largely dependent on the activity of the ATPase enzyme (Cartwright et al., “Effect of Ethanol on Activity of the plasma-membrane ATPase in, and Accumulation of Glycine by, Saccharomyces cerevisiae,” J Gen Microbiology 133:857-865 (1987), which is hereby incorporated by reference in its entirety). Inhibition of the enzyme decreases the transport of sugar substrates and nutrients across the cell membrane and thus affects the growth and fermentation ability of the cell (D'Amore et al., “A Study of Ethanol Tolerance in Yeast,” Crit Rev Biotechnology 9(4):287-304 (1990), which is hereby incorporated by reference in its entirety). On this basis, it stands to reason that cells with an increased content of PI, as in the case of opi1, would have a higher resistance to ethanol. Additionally, altering the ratio of charged head groups has been discussed as a possible mechanism for increased ethanol tolerance (Clark et al., “Altered Phospholipids Composition in Mutants of Escherichia coli Sensitive or Resistant to Organic Solvents,” J Gen Microbiology 113:267-274 (1979), which is hereby incorporated by reference in its entirety). Since PI is the major anionic phospholipid in yeast, it is possible that alterations in membrane charge are responsible for the increased tolerance in PI-enriched cells.

Evaluating growth in the presence of exogenous ethanol has been used extensively as a means of determining ethanol tolerance (Aguilera et al., “Relationship between Ethanol Tolerance, H+-ATPase Activity and the Lipid Composition of the Plasma Membrane in Different Wine Yeast Strains,” Int J Food Microbiology 110:34-42 (2006); Kalmokoff et al., “Evaluation of Ethanol Tolerance in Selected Saccharomyces Strains,” ASBC J 43(4):189-196 (1985); and You et al., “Ethanol Tolerance in the Yeast Saccharomyces cerevisiae is Dependent on Cellular Oleic Acid Content,” Applied Environ Microbiology 69:1499-1503 (2003), which are hereby incorporated by reference in their entirety). Although the response of a given yeast strain to exogenously imposed ethanol may not mirror the response of the same yeast to endogenously produced ethanol, this method provides a relatively quick means of gauging the ethanol tolerance of a strain. It is especially useful when dealing with ethanol concentrations that are higher than the concentration naturally produced by the organism when, for example, investigating the effects of ethanol on the cell.

The following conclusions can be made regarding the effect of the opi1 mutation on ethanol tolerance. Cells containing the opi1 mutation exhibited higher tolerance to exogenous ethanol, in terms of cell viability, at ethanol concentrations that drastically affect the viability of the wild type cell. The tolerance of opi1 in 15% and 18% ethanol is further increased when the yeast is grown in the presence of inositol. The opi1 strain, with the ability to constitutively produce inositol regardless of media composition, showed less inhibition of cell growth in the presence of ethanol than the wild type strain, particularly in inositol-free media.

Inositol, PI, and phosphorylated forms of PI are essential components of many cellular processes. Phosphoinositides play an important role in membrane transport and lipid signaling. Species of phosphoinositides are specifically localized in the cell, providing signaling functions for various organelles, including the plasma membrane (Jesch et al., “Yeast Inositol Phospholipids: Synthesis, Regulation, and Involvement in Membrane Trafficking and Lipid Signaling,” In: Cell Biology and Dynamics of Yeast Lipids, G. Daum (Ed.). Research Signpost, Kerala, India, Vol: 37/661: 105-131 (2005), which is hereby incorporated by reference in its entirety). Inositol synthesis is meticulously controlled in the yeast cell (Greenberg et al., “Genetic Regulation of Phospholipid Biosynthesis in Saccharomyces cerevisiae,” Microbiological Reviews 60(1):1-20 (1996), which is hereby incorporated by reference in its entirety).

Yeast cells containing a higher concentration of phosphatidylinositol (PI) in the cellular membrane, due to inositol supplementation in the growth media, have been shown to tolerate and produce higher concentrations of ethanol (Chi et al., “Role of Phosphatidylinositol (PI) in Ethanol Production and Ethanol Tolerance By a High Ethanol Producing Yeast,” J Industrial Microbiology and Biotechnology 22:58-63 (1999); and Furukawa et al., “Effect of Cellular Inositol Content on Ethanol Tolerance of Saccharomyces cerevisiae in Sake Brewing,” J Bioscience Bioengineering 98(2):107-113 (2004), which are hereby incorporated by reference in their entirety). In these reports, increasing cellular inositol content through media supplementation had the following effects on yeast cells: increased cell viability, decreased nutrient leakage, higher PI concentrations and subsequent higher ATPase activity when compared to cultures with limited inositol availability (Chi et al., “Role of Phosphatidylinositol (PI) in Ethanol Production and Ethanol Tolerance By a High Ethanol Producing Yeast,” J Industrial Microbiology and Biotechnology 22:58-63 (1999); and Furukawa et al., “Effect of Cellular Inositol Content on Ethanol Tolerance of Saccharomyces cerevisiae in Sake Brewing,” J Bioscience Bioengineering 98(2):107-113 (2004), which are hereby incorporated by reference in their entirety). Ethanol production has also been increased in some strains by inositol supplementation (Chi et al., “Role of Phosphatidylinositol (PI) in Ethanol Production and Ethanol Tolerance By a High Ethanol Producing Yeast,” J Industrial Microbiology and Biotechnology 22:58-63 (1999), which is hereby incorporated by reference in its entirety)). These reports, in addition to the present invention clearly demonstrate the importance of cellular inositol and/or PI content in conferring ethanol tolerance in yeast.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of producing organisms tolerant to alcohol, said method comprising:
selecting a microorganism needing tolerance to alcohol and
modifying the selected microorganism under conditions effective to overproduce inositol by the microorganism compared to when the microorganism is not modified, said modified microorganism being tolerant to alcohol.

2. The method of claim 1, wherein the microorganism is a species of yeast.

3. The method of claim 2, wherein the yeast is selected from the group consisting of a Pichia species, a Candida species, a Schizosaccharomyces species, and a Saccharomyces species.

4. The method of claim 3, wherein said microorganism is Saccharomyces cerevisiae.

5. The method of claim 1, wherein said modifying is carried out by inactivation or deletion or substitution of a selected gene that prevents the overproduction of inositol.

6. The method of claim 5, wherein said modifying is carried out by transforming the microorganism with a nucleic acid construct used to prevent gene expression of the selected gene, said construct comprising:
a 5′ DNA promoter sequence;
a nucleic acid molecule that inactivates the selected gene which prevents the overproduction of inositol; and
a 3′ terminator sequence, wherein the 5′ DNA promoter sequence and the 3′ terminator sequence are operatively coupled to the nucleic acid molecule.

7. The method of claim 5, wherein said nucleic acid molecule comprises a nucleotide sequence encoding part or all of the selected gene in anti-sense orientation.

8. The method of claim 5, wherein said nucleic acid molecule comprises a nucleotide sequence encoding part or all of the gene in anti-sense orientation followed by a nucleotide sequence encoding part or all of the gene in sense orientation.

9. The method of claim 5, wherein said selected gene is OPI1.

10. The method of claim 1, wherein said modifying includes overexpression of a gene encoding a protein in the inositol biosynthesis pathway.

11. The method of claim 1, wherein the microorganism is Saccharomyces cerevisiae with its INO1 gene being overexpressed or constitutively expressed.

12. The method of claim 1 further comprising:
combining the growth of the microorganism with an inositol-supplemented media.

13. The method of claim 1, wherein the modified microorganism is tolerant to high osmotic shock.

14. A method of producing alcohol, said method comprising:
providing a microorganism tolerant to alcohol, said microorganism being modified to overproduce inositol by the microorganism compared to when the microorganism is not modified and
treating a fermentable feedstock with the modified microorganism under conditions effective to produce alcohol.

15. The method of claim 14, wherein the fermentation product is ethanol and CO₂.

16. The method of claim 15, wherein said microorganism is a species of yeast.

17. The method of claim 16, wherein the yeast is selected from the group consisting of a Pichia species, a Candida species, a Schizosaccharomyces species, and a Saccharomyces species.

18. The method of claim 17, wherein said microorganism is Saccharomyces cerevisiae.

19. The method of claim 15, wherein the microorganism is modified by inactivation or deletion or substitution of a selected gene that prevents the overproduction of inositol.

20. The method of claim 19, wherein said inactivation is carried out by transforming the microorganism with a nucleic acid construct used to prevent gene expression, said construct comprising:
a 5′ DNA promoter sequence;
a nucleic acid molecule that causes inhibition of inositol biosynthesis; and
a 3′ terminator sequence, wherein the 5′ DNA promoter sequence and the 3′ terminator sequence are operatively coupled to the nucleic acid molecule.

21. The method of claim 19, wherein said nucleic acid molecule comprises a nucleotide sequence encoding part or all of the selected gene is in anti-sense orientation.

22. The method of claim 21, wherein said nucleic acid molecule comprises a nucleotide sequence encoding part or all of the selected gene in anti-sense orientation followed by a nucleotide sequence encoding part or all of the selected gene in sense orientation.

23. The method of claim 21, wherein said selected gene is OPI1.

24. The method of claim 15, wherein the microorganism is modified to overexpress a gene encoding a protein in the inositol biosynthesis pathway.

25. The method of claim 15, wherein the microorganism is Saccharomyces cerevisiae with its INO1 gene being overexpressed or constitutively expressed.

26. The method of claim 15, wherein said fermentable feedstock is supplemented with inositol.

27. The method of claim 15, wherein said fermentable feedstock is from starches, sugars, or lignocellulosic materials.

28. The method of claim 15, wherein said fermentable feedstock is selected from the group consisting of corn, trees, grasses, hemp, and sugarcane.

29. The method of claim 14, wherein the modified microorganism is tolerant to high osmotic shock.
